(19) United States
(12) Patent Application Publication
(10) Pub. No.: US 2023/0331070 A1
(43) Pub. Date: Oct. 19, 2023

(54) Vehicle Fragrance Dispensing Device

(71) Applicant: Pura Scents, Inc., Pleasant Grove, UT (US)

(72) Inventors: Caleb Probst, Nephi, UT (US); Christopher Michael Cooper, Saratoga Springs, UT (US); Clint Cook, American Fork, UT (US); David Freitag, Pleasant Grove, UT (US); Hannah Trimble, Salt Lake City, UT (US); Juan Carlos Altuna, South Jordan, UT (US); Michael Baird, Pleasant Grove, UT (US); Michaylo Chubak, West Valley City, UT (US); Jared Raulston, Pleasant Grove, UT (US); Auston Tesch, Vineyard, UT (US); Mason Grant, Payson, UT (US); Fnu Mayadevi, Pleasant Grove, UT (US); Trevor Davis, American Fork, UT (US)

(21) Appl. No.: 18/157,624

(22) Filed: Jan. 20, 2023

Related U.S. Application Data
(60) Provisional application No. 63/331,752, filed on Apr. 15, 2022.

(51) Int. Cl. B60H 3/00

(52) U.S. Cl. CPC B60H 3/0035; B60H 2003/0042; B60H 2003/0064

(57) ABSTRACT

A vehicle scent dispensing device may comprise a scent cartridge configured to contain a scenting agent, a controller configured to determine a scent diffusion level of the scenting agent, a valve, and a fan. The controller may be configured operate the valve and control the fan to regulate an airflow pathway to the scenting agent and to draw air through the airflow pathway and over the scenting agent based on the scent diffusion level.

### BACKGROUND

The present disclosure relates to scent dispensation.

Existing solutions for dispensing scents within a vehicle include conventional car air fresheners that have a scented oil contained on a blotting paper structure that hangs in the car. The scented oil emits the scent within the car as the conventional car air freshener hangs. However, the scent of the conventional car air freshener cannot be evenly dispersed and over time the scented oil loses its scent strength. In other existing solutions, car scent dispensers include liquid scented oil vials that can be opened to expose the liquid scent oil and disperse the scent within the vehicle. However, the temperatures within a vehicle can fluctuate between hot and cold temperatures and these variations in temperatures affect the liquid scented oil in the conventional car air fresheners. In high temperatures, the scent can be dispersed too quickly and create overpowering scents, along with evaporation of the liquid scented oil. While in low temperatures, the scent does not diffuse as well.

In addition these solutions are limited as it is often difficult to tell when a fragrance dispenser has run out of fragrance or when a hanging paper air freshener is running out of scent, as the dispenser typically lacks any sort of indicator and one is left to smell the dispenser manually to determine how much fragrance is actually left, which is ineffective as the dispenser itself may still smell like the diffused fragrance up close, but that diffused fragrance may have little effect in the vehicle that is to be perfumed.

### SUMMARY

According to one innovative aspect of the subject matter being described in this disclosure, an example system includes a vehicle scent dispensing device. The vehicle scent dispensing device also includes a scent cartridge configured to contain a scenting agent; a controller configured to determine a scent diffusion level of the scenting agent; a valve, wherein the controller is configured to operate the valve to regulate an airflow pathway to the scenting agent based on the scent diffusion level; and a fan, wherein the controller is configured to control the fan to draw air through the airflow pathway and over the scenting agent based on the scent diffusion level.

According to one innovative aspect of the subject matter being described in this disclosure, an example system includes a vehicle scent dispensing device. The vehicle scent dispensing device also includes a motion sensor that captures motion data; a temperature sensor that captures temperature data, a diffusion device that determines a diffusion level of a scent cartridge based on the motion data and the temperature data, a valve mechanisms that opens an airflow pathway through the diffusion device based on the diffusion level, and a fan that causes air to pass through the airflow pathway and over the scent cartridge.

However, this list of features and advantages is not all-inclusive and many additional features and advantages are within the scope of the present disclosure. Moreover, it should be noted that the language used in the present disclosure has been principally selected for readability and instructional purposes, and not to limit the scope of the subject matter disclosed herein.

### BRIEF DESCRIPTION OF THE DRAWINGS

FIG. 1A is a block diagram illustrating an example system for vehicle scent dispensation.

FIG. 1B is a block diagram illustrating an example vehicle scent dispenser device.

FIG. 2A is a bottom perspective view of an example vehicle scent dispenser device.

FIG. 2B is a top perspective view of the example vehicle scent dispenser device.

FIG. 2C is a right side view of the example vehicle scent dispenser device.

FIG. 2D is a rear view of the example vehicle scent dispenser device.

FIG. 2E is a bottom view of the example vehicle scent dispenser device.

FIG. 2F is a front view of the example vehicle scent dispenser device.

FIG. 2G is a top view of the example vehicle scent dispenser device.

FIG. 2H is a left side view of the example vehicle scent dispenser device.

FIG. 3 is a cross-sectional view of the example vehicle scent dispenser device.

FIG. 4A depicts an example scent cartridge with a valve recess configured to accommodate a valve.

FIGS. 4B and 4C depict various views of a valve

FIG. 4D depicts a scent cartridge with the valve removed.

FIG. 5A depicts an example scent cartridge including a sliding plate valve.

FIG. 5B depicts an exploded view of the example scent cartridge depicted in the example of FIG. 5A.

FIG. 5C depicts an example sliding plate valve.

FIG. 6A depicts an example vehicle scent dispensing device including a rotatable plate valve.

FIG. 6B depicts a partially exploded view of an example vehicle scent dispensing device including a rotatable plate valve.

FIG. 7 is a flowchart of an example method for activating a vehicle scent dispenser device.

### DETAILED DESCRIPTION

The technology described in this disclosure relates to dispensing scent within a vehicle using a vehicle scent dispensing device. As an example, the technology allows a vehicle scent dispensing device to be positioned in a vehicle, such as a car or truck and dispense a scent in an efficient manner that takes the state of the vehicle (e.g., moving, parked, etc.) and the temperature inside of the vehicle into account. By accounting for when the state of the vehicle and the temperature inside of the vehicle, the vehicle scent dispensing device is able to create a stable and consistent scenting experience for a user while also prolonging the life of the scent cartridge compared to conventional scent systems.

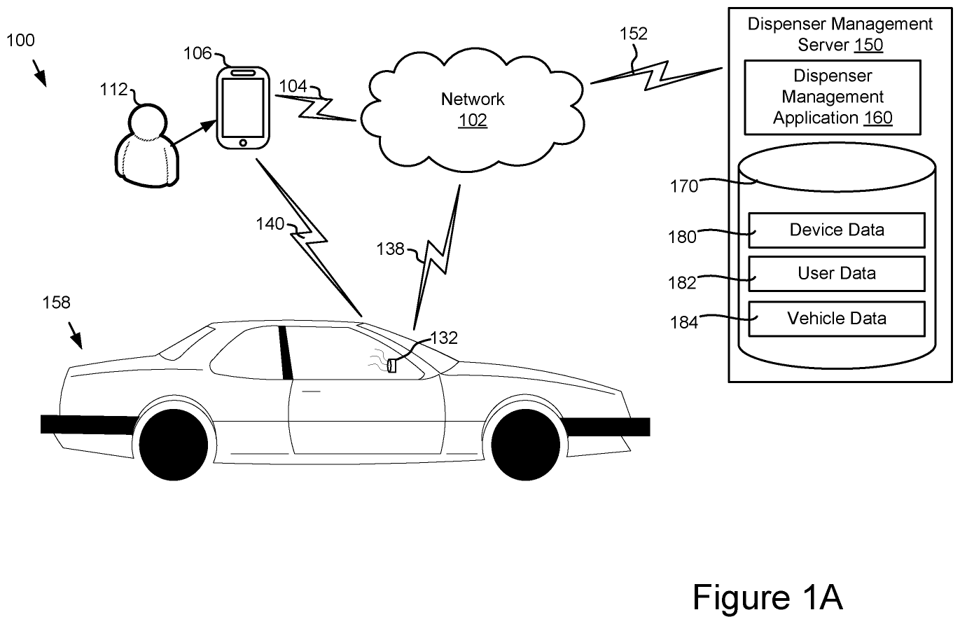

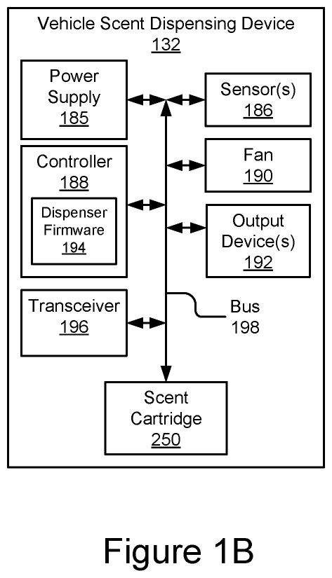

FIG. 1A is a block diagram illustrating an example system 100 for vehicle scent dispensation. The system 100 may include one or more vehicle scent dispensing devices 132 positioned within a vehicle 158. In a typical implementation, a single vehicle scent dispensing device 132 is positioned within a single vehicle 158, however in large vehicles, such as buses or trains, multiple vehicle scent dispensing devices 132 may be positioned throughout the large vehicle for effective scent dispensing. In some implementations, the vehicle scent dispensing device 132 may be positioned by a user in various locations and may stay positioned using various attachment mechanisms, such as magnets, hook and loop fasteners, clips, etc. For example, in one implementation, the device 132 may have a magnet on a back surface that can attach to any appropriate surface, or a mounting plate can be positioned on a surface for the device 132 to mount to. The magnet allows the device 132 to be portable and moved as needed while operating using battery power.

As shown, system 100 further includes user device 106 and a dispenser management server 150, which are electronically communicatively coupled via a network 102 for interaction with one another and the scent dispenser(s) 132 using standard networking protocols, as reflected by signal lines 104, 138, and 152. In some implementations, the vehicle scent dispensing device 132 may instead be communicatively coupled to the user device 106, such as by Bluetooth, beacons, or other networking protocols as depicted by signal line 140, to allow the vehicle scent dispensing device 132 to connect with a paired user device 106 when in proximity rather than through the network 102.

The dispenser management application 160 operable by the dispenser management server 150 can receive operational data from the vehicle scent dispensing device 132 in association with the vehicle 158, user device 106, and/or user(s) 112, with which they are associated. The dispenser management application can receive management requests for a vehicle scent dispensing device 132, such as a diffusion level or various data that can be analyzed over time to improve the scent dispensing experience for the user 112.

The dispenser management server 150 includes a data store 170 storing various types of data used by the dispenser management application 160. Example data types include device data 180, user data 182, and/or vehicle data 184. The device data 180 may include a device model, a scent cartridge type, usage statistics, scent diffusion time, temperature variations, etc. The user data 182 may include entries for the users 112 of the system 100. A given entry may include a unique identifier for the user, a unique identifier for the user device 106, contact information for the user (e.g., address, phone number, electronic address (e.g., email)), payment information, scent subscription information specifying which reoccurring scent cartridges 250 should be shipped to the user, etc. The vehicle data 184 may include entries for the data related to the vehicle, such as vehicle type, average detected internal and/or external vehicle temperatures, movement data, geolocation data from the user device 106, etc.

FIG. 1B is a block diagram illustrating example components of a vehicle scent dispenser device 132, which is depicted as including a power supply 185, one or more sensor(s) 186, a controller 188, output device(s) 192, dispenser firmware 194, a fan 190, and any number of scent cartridge(s) 250. The components 185, 186, 188, 190, 192, and 250 are communicatively coupled via a communications bus 198. The controller 188 may include a non-transitory memory device, or may be coupled to a non-transitory memory device also coupled for communication via the bus 198. The non-transitory memory device may store software that specially configures the controller, such as the dispenser firmware 194. The power supply 185 may be any AC and/or DC power supply for powering the vehicle scent dispensing device 132. In some implementations, the power supply 185 may be a battery and may be configured to charge when plugged into an AC and/or DC power supply. The controller 188 may be a microchip that controls the constituent electronics (e.g., sensor(s) 186, output device(s) 192, fan 190, etc.) of the vehicle scent dispensing device 132.

The one or more sensor(s) 186 may include one or more temperature sensors for detecting the ambient temperature adjacent to the vehicle scent dispensing device 132. For example, when the vehicle scent dispensing device 132 is positioned within the vehicle 158, the temperatures inside of the vehicle 158 can widely fluctuate depending on the season, location of the vehicle 158 (e.g., parked in shade or sun), if the vehicle 158 is running, if the climate controls in the care are operating, etc. The temperature sensors can detect these various temperatures and the vehicle scent dispensing device 132 can use those readings to determine efficient diffusion of the scent.

The one or more sensor(s) 186 may further include one or more accelerometers or other movement sensors for detecting movement of the vehicle scent dispensing device 132. For example, the accelerometers or other movement sensors can detect when the scent dispensing device 132, and consequently the vehicle 158 in which the scent dispensing device is located, is moving compared to when scent dispensing device 132 and vehicle 158 is stationary. The vehicle scent dispensing device 132 can use the movement data for efficient diffusion of the scent.

In some implementations, the one or more sensor(s) 186 may further include a cartridge sensor for sensing when to replace a scent cartridge(s) 250 installed in the vehicle scent dispensing device 132, optical sensor(s) for detecting an identity of scent cartridge(s) 250 installed in the vehicle scent dispensing device 132, ambient light sensor to detect a light level in a surrounding environment (e.g., vehicle 158), and/or a motion sensor to detect motion of objects (e.g., user 112 or other people) in the surrounding environment, etc. In some embodiments, the sensors 186 may include a separate cartridge bay temperature sensor for the scent cartridge 250 (e.g., that measures temperatures at the scent cartridge 250 to be used to adjust a speed and/or frequency of the fan 190).

The vehicle scent dispensing device may also include a transceiver 196 having a wireless interface configured to communicate with the devices coupled to the network 102, such as the dispenser management server 150, and/or other components of the network 102 using standard communication protocols, such as Internet protocols. Further, the transceiver 196 may be configured to wirelessly transmit data via a network to connect to other devices, such as the user device 106. By way of further example, the transceiver 196 may transmit data to the user device 106 to which it is linked using a protocol compliant with IEEE 802.15, such as Zigbee®, Z-Wave®, Bluetooth®, or another suitable standard. Further embodiments are also possible and contemplated. In some embodiments, the transceiver 196 may be embedded in the controller 188 or may be a component distinct from the controller and coupled to the controller 188 via the bus 198.

The output device(s) 192 may include light sources and/or audio reproduction devices, although further suitable output devices are also contemplated and applicable. In some implementations, the light sources and/or audio reproduction devices may be controlled to produce output consistent with a scent being emitted by the scent dispenser (e.g., a low, soothing light and music may be output in conjunction with a relaxing scent being emitted), or to communicate various alerts, such as low power, low scent cartridge levels, etc.

The fan 190 may include a motor that has one or more fan blades that force air through the device 132 when the motor is operating. The fan 190 may operate at various speeds based on how quickly or slowly the motor runs. The fan 190 may be configured to nest within a housing of the device 132 and cause air to move through an airflow pathway over the scent cartridges 250 for scent diffusion. In some implementations, the fan 190 may be able to turn on and off as signaled by the controller 188 which results in substantially immediate scent diffusion.

The scent cartridge 250 may be removable and include a scenting agent, such as gel, oil, oil soaked paper, or the like, that diffuses a scent into the nearby air. The scent cartridges 250 may be replaceable when the scent is diminished and new scent cartridges 250 can be inserted into the device 132. The scent cartridges 250 may have various scent profiles and information about the scent cartridges 250 may be stored in the dispenser management application 160 to store the various scent profiles, ages of the scent cartridges 250, duration of use of the scent cartridges 250, exposed temperatures of the scent cartridges 250, etc.

Returning to FIG. 1A, the user device 106 may be a computing device having data processing and communication capabilities. In some embodiments, a user device 106 may include a processor (e.g., virtual, physical, etc.), a memory, a power source, a network interface, and/or other software and/or hardware components, such as a display, graphics processor, wireless transceivers, keyboard, camera, sensors, firmware, operating systems, drivers, various physical connection interfaces (e.g., USB, HDMI, etc.).

The user device 106 may couple to and communicate with one another and the other entities of the system 100 via the network 102 using a wireless and/or wired connection. Examples of user device 106 may include, but are not limited to, mobile phones (e.g., feature phones, smart phones, etc.), tablets, smartwatches or other smart wearables, laptops, desktops, netbooks, server appliances, servers, virtual machines, TVs, set-top boxes, media streaming devices, portable media players, navigation devices, personal digital assistants, car access panels, etc. In addition, while a single user device 106 is depicted in FIG. 1A, it should be understood that any number of user devices may connect to and communicate with other components of system 100.

The user device 106 may include a scent application, which allows the user to set vehicle scent dispensing device 132 settings, turn scent dispensing device 132 on and off, purchase scent cartridges 250 for the vehicle scent dispensing device 132, set up a vehicle scent dispensing device 132, register an account, set up a vehicle profile, associate a vehicle scent dispensing device 132 with a particular vehicle and/or vehicle profile, view analytics reflecting the user's historical use of his/her scent vehicle scent dispensing device 132, enable user profiles to use and setup scent profiles for the scent dispensing device installed in the vehicle, set a profile hierarchy (e.g., set which user profile(s) is/are the dominant user profile), etc.

The dispenser management server 150 may include one or more computing devices having data processing, storage, and communication capabilities. For example, the server 150 may include one or more hardware servers, virtual servers, server arrays, storage devices and/or systems, etc., and/or may be centralized or distributed/cloud-based. In some embodiments, the server 150 may include one or more virtual servers, which operate in a host server environment and access the physical hardware of the host server including, for example, a processor, memory, storage, network interfaces, etc., via an abstraction layer (e.g., a virtual machine manager).

While not depicted, the server 150 may include a (physical, virtual, etc.) processor, a non-transitory memory, a network interface, and a data store 170, which may be communicatively coupled by a communications bus. Similarly, the user device 106 may include a physical processor, a non-transitory memory, a network interface, a display, an input device, a sensor, and a capture device. It should be understood that the server and the client device may take other forms and include additional or fewer components without departing from the scope of the present disclosure.

Software operating on the server 150 (e.g., the dispenser management application 160, an operating system, device drivers, etc.) may cooperate and communicate via a software communication mechanism implemented in association with a server bus. The software communication mechanism can include and/or facilitate, for example, inter-process communication, local function or procedure calls, remote procedure calls, an object broker (e.g., CORBA), direct socket communication (e.g., TCP/IP sockets) among software modules, UDP broadcasts and receipts, HTTP connections, etc. Further, any or all of the communication could be secure (e.g., SSH, HTTPS, etc.).

As shown, the server 150 may include a dispenser management application 160 embodying a remotely accessible scent service. The dispenser management application 160 may send data to and receive data from the other entities of the system including the controllers 188, the user device 106, etc. The dispenser management application 160 may be configured to store and retrieve data from one or more information sources, such as the data store 170. In addition, while a single server 150 is depicted in FIG. 1A, it should be understood that one or more servers 150 may be included.

In some embodiments, the dispenser firmware 194, the scent application, the dispenser management application 160, etc., may require users to be registered to access the acts and/or functionality provided by them. For example, to access various acts and/or functionality provided by the scent application, dispenser management application 160, and/or scent dispensers 132, these components may require a user to authenticate his/her identity (e.g., by confirming a valid electronic address). In some instances, these entities 132, 160, etc., may interact with a federated identity server (not shown) to register/authenticate users. Once registered, these entities 132, 160, etc., may require a user seeking access to authenticate by inputting credentials in an associated user interface.

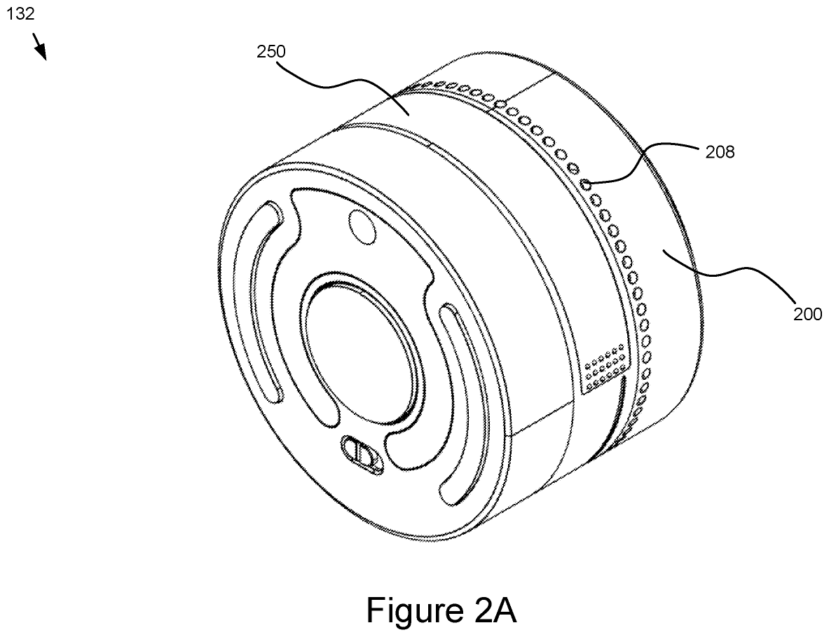

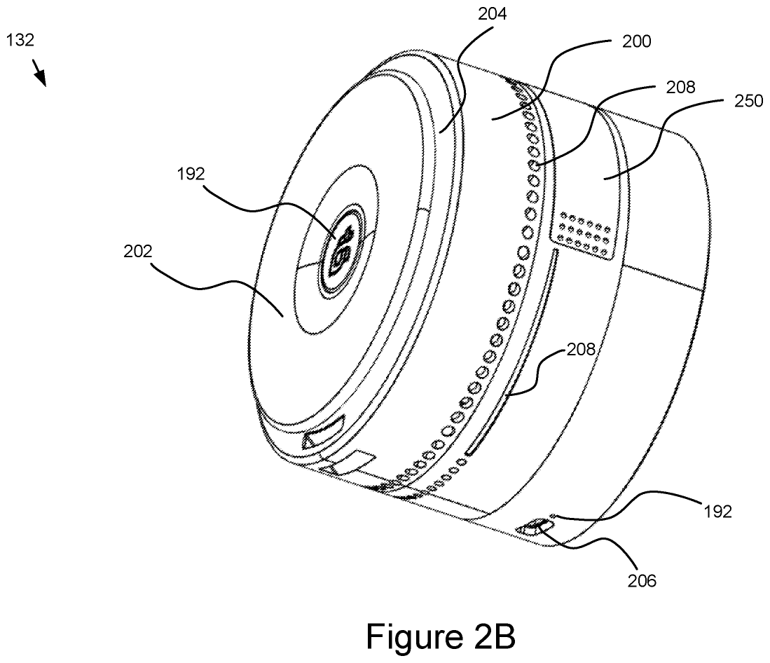

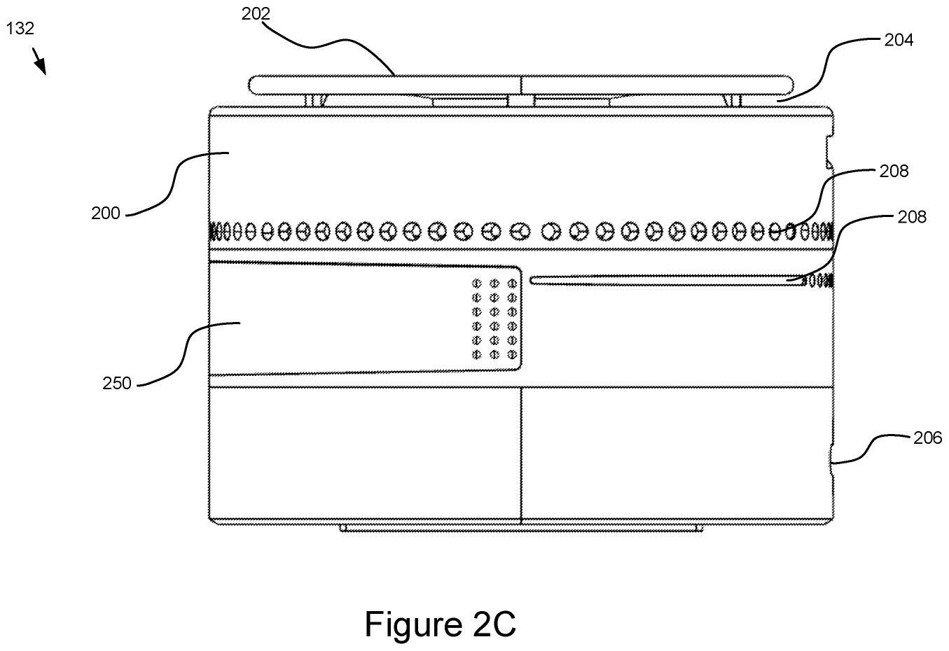

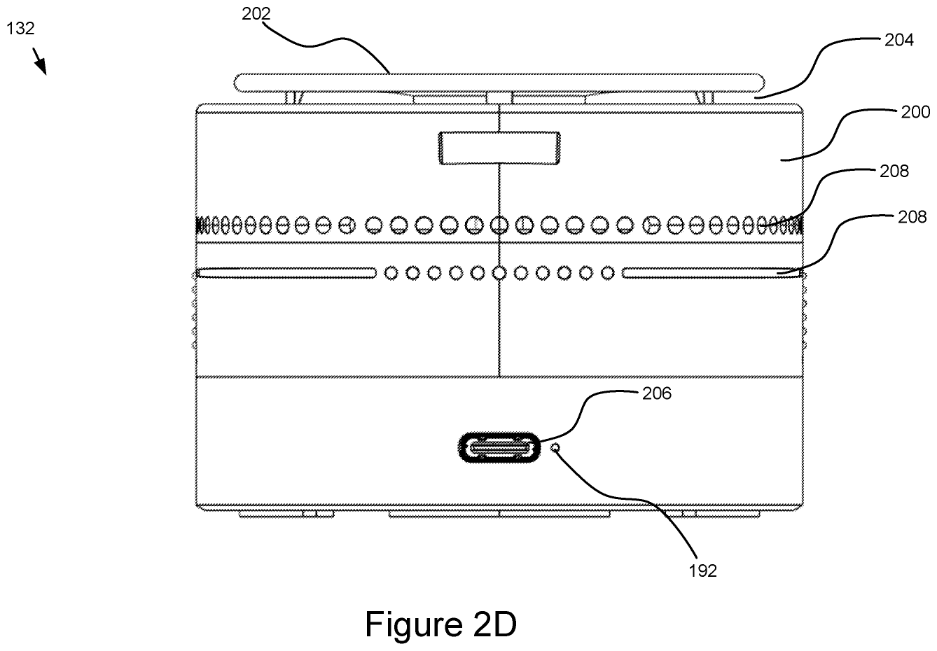

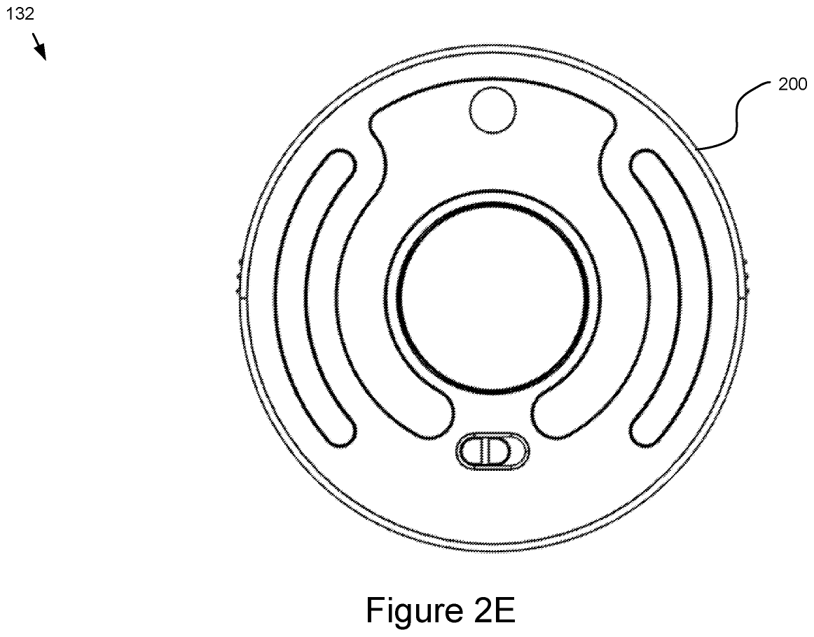

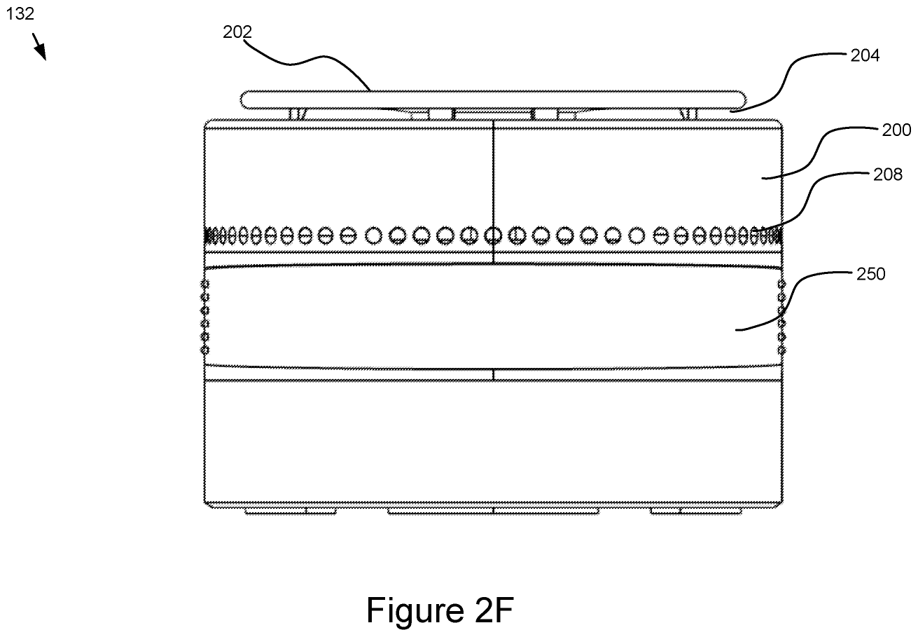

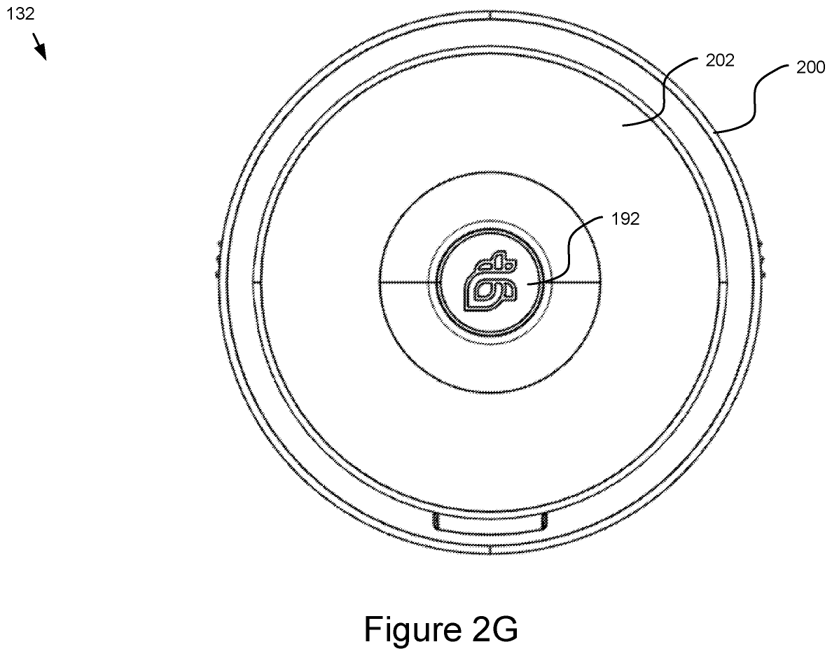

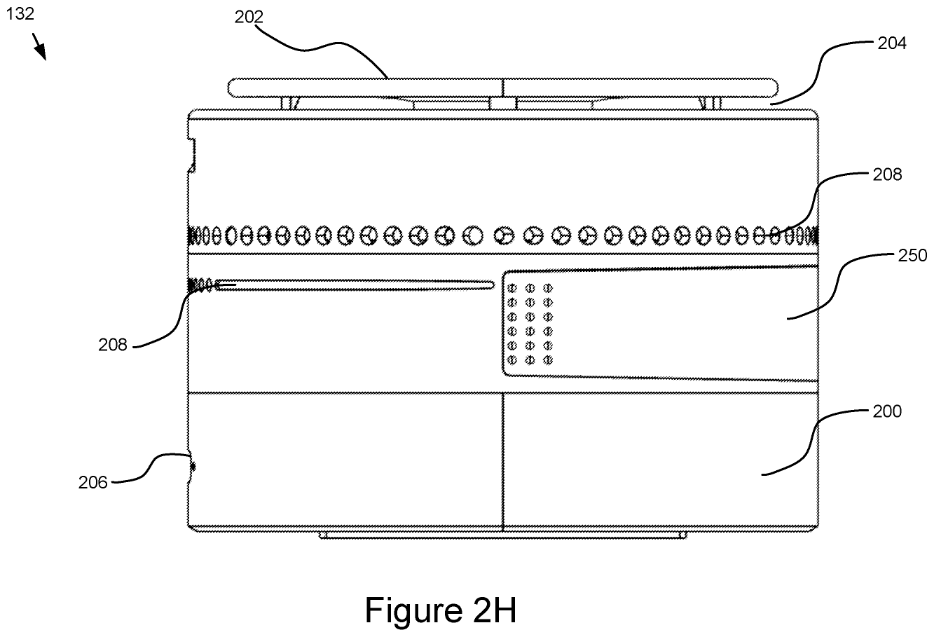

FIGS. 2A-2H are various views of an example vehicle scent dispensing device 132. As shown, the vehicle scent dispensing device 132 includes a housing 200 that houses the components of the scent dispenser (e.g., scent cartridge 250, fan 190, power supply 185, sensor 186, etc.). In some implementations, as shown, the device 132 may be cylindrical in shape with flat top and bottom surfaces, however, other shapes and configurations are also contemplated. The housing 200 includes a top surface 202 and a recess 204 through which scent is dispersed from within the housing 200.

As shown in FIG. 2C, the housing 200 may include perforations 208 through which air can be drawn in and/or out of the housing 200 and then blown out of the recess 204 via the fan 190 (not shown). In some implementations, a portion of the housing 200 that includes the perforations 208 may be rotatable about an axis to allow for air to flow through an airflow pathway of the device 132. In this example, a user can manually rotate that portion of the housing and allow holes in the interior of the housing 200 to line up with exterior holes that allow air through an airflow pathway the device 132 for scent diffusion. In some implementations, the rotatable portion may rotate about a bearing so the portion can be rotated without wear and the rotation acts as a swivel mechanism to turn the scent diffusion on and off. In some implementations, these holes can be sealed by rotating the portion of the housing 200 to a closed position and/or opened by rotating the portion of the housing 200 to an open position. In some implementations, the housing 200 can be further sealed in the closed position using various O-rings or other sealing devices to halt the airflow.

As shown in FIG. 2D, the housing 200 may include an electronic port 206, such as USB-C, USB, or other electronic port, which the user can utilize to charge one or more batteries of the power supply 185, connect to other devices, plug the scent dispenser into a computer to update firmware, set settings, etc. The housing 200 may also include one or more output devices 192, such as LED(s) or a speaker.

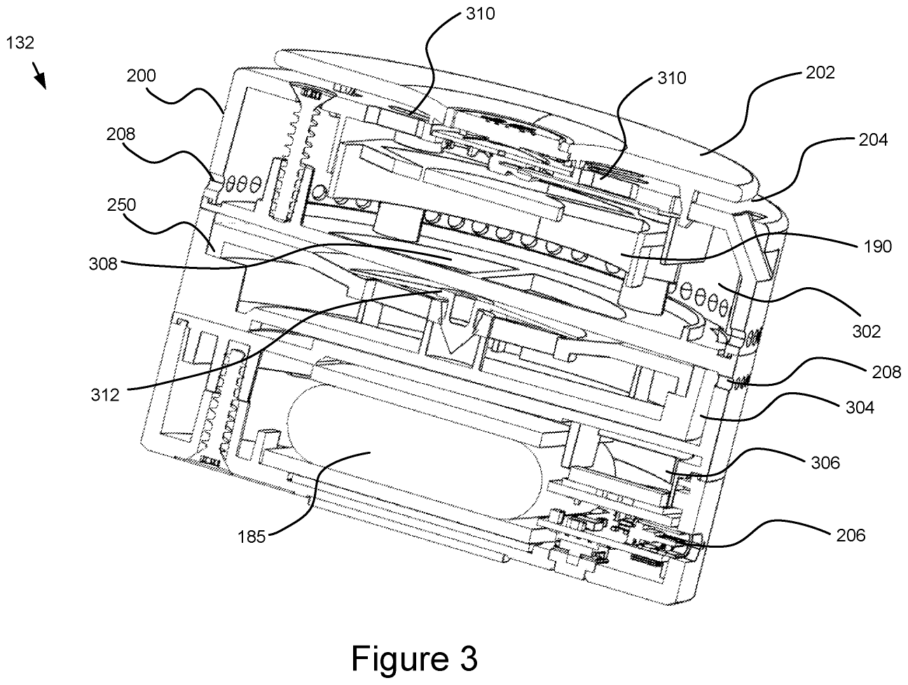

FIG. 3 depicts a cross-sectional view of the interior of the vehicle scent dispensing device 132 according to a first embodiment. As shown in the example of FIG. 3, the housing 200 forms a fan compartment 302, a scent cartridge compartment 304, and a control component compartment 306. In various embodiments, the compartments 302, 304, and 306 may be combined or further divided into any number of compartments. In the example of FIG. 3, the scent cartridge compartment 304 and the fan compartment 302 are connected by opening 308 to allow scent from the scent cartridge 250 to be drawn by fan 190 from the scent cartridge compartment 302 through the fan compartment 302 and out of vent(s) 310 into the vehicle. When in operation, fan 190 draws air into the vehicle scent dispensing device 132 through perforations 208. This creates a pressure differential between the fan compartment 302 and the interior of the scent cartridge 250. The pressure differential causes a valve 312 on the scent cartridge 250 to open and allow scent to flow from the scent cartridge. Differing fan speeds and/or frequencies cause the valve 312 to open to a different degree and therefore the amount of scent dispersed can be regulated by adjusting the speed and/or frequency of the fan. For example, at higher fan speeds, a greater pressure differential between the interior of the scent cartridge 250 and the scent cartridge compartment 304 is created and the valve 312 opens wider to allow more scent to be dispersed. When fan 190 is not in operation, valve 312 remains closed and creates a barrier to the interior of the scent cartridge 250 to prolong the life of the scent cartridge 250 and to prevent scent from being emitted by vehicle scent dispensing device 132 when the device is not in operation.

In some implementations, the scent cartridge compartment 304 may include one or more spring-loaded pins that can detect when a scent cartridge 250 is positioned within the scent cartridge compartment 304. In further implementations, these spring-loaded pins can also have various configurations depending on what type of scent cartridge 250 is installed. For example, the spring-loaded pins may create an electrical connection between scent cartridge 250 and controller 188 such that controller 188 can determine the characteristics of scent cartridge 250 (e.g., the scent contained in scent cartridge 250, the amount of time scent cartridge 250 has been installed in the vehicle scent dispensing device 132, the amount of time the scent cartridge 250 has been active, etc.).

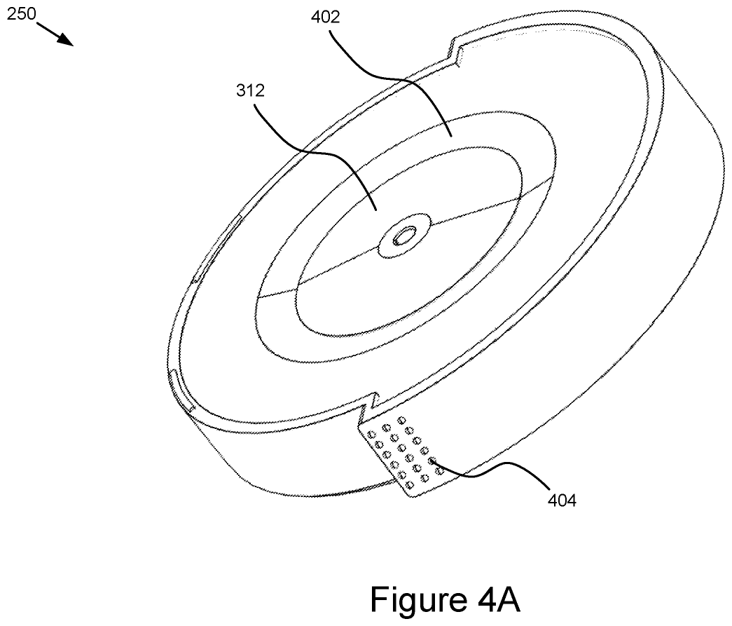

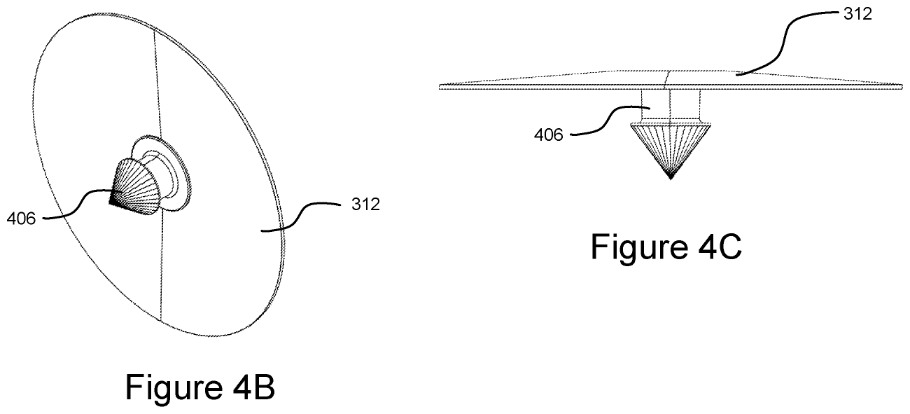

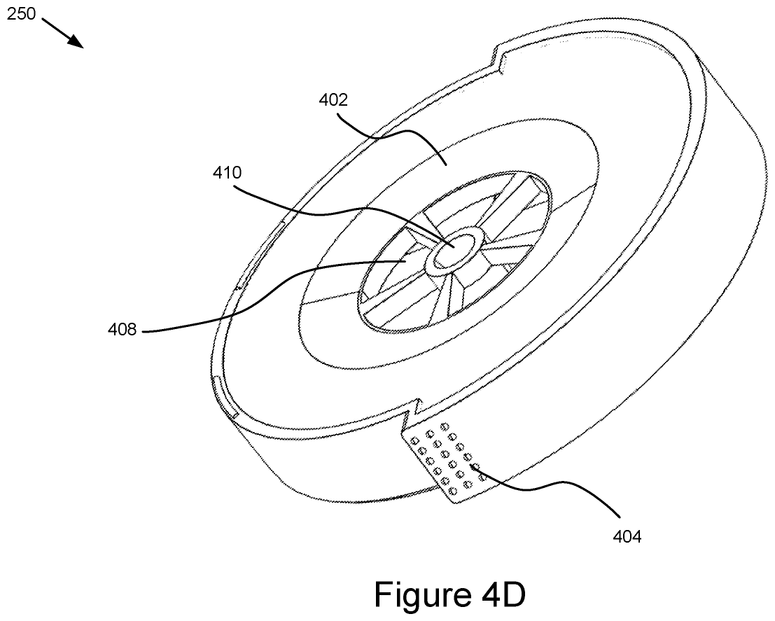

FIGS. 4A-4D depict various views of an example scent cartridge 250 for the example vehicle scent dispensing device 132. An example scent cartridge 250 with a valve recess 402 configured to accommodate valve 312 is depicted in FIG. 4A. In some implementations, scent cartridge 250 includes protrusions 404 on the sides to provide additional grip for handling the scent cartridge 250.

FIGS. 4B and 4C depict various views of a valve 312, such as a semi flexible umbrella valve formed out of silicone, suitable for use in scent cartridge 250 according to some implementations. As described above, the valve 312 may be configured to seal against an edge of the valve recess 402 when the fan 190 is not operating to close off the scent cartridge 250 from outside air flow and retain a seal with the valve recess 402. The valve 312 includes a pin 406 configured to secure the valve 312 to the scent cartridge 250.

FIG. 4D depicts a scent cartridge 250 with the valve 312 removed. As depicted in the example of FIG. 4D, the scent cartridge includes one or more outlets 408 configured to expose the scenting agent of scent cartridge 250 to an airflow pathway when valve 312 is opened and allow scent to diffuse from the scent cartridge 250. Scent cartridge 250 includes a socket 410 configured to retain pin 406 of valve 312 to hold valve 312 in place in the valve recess 402.

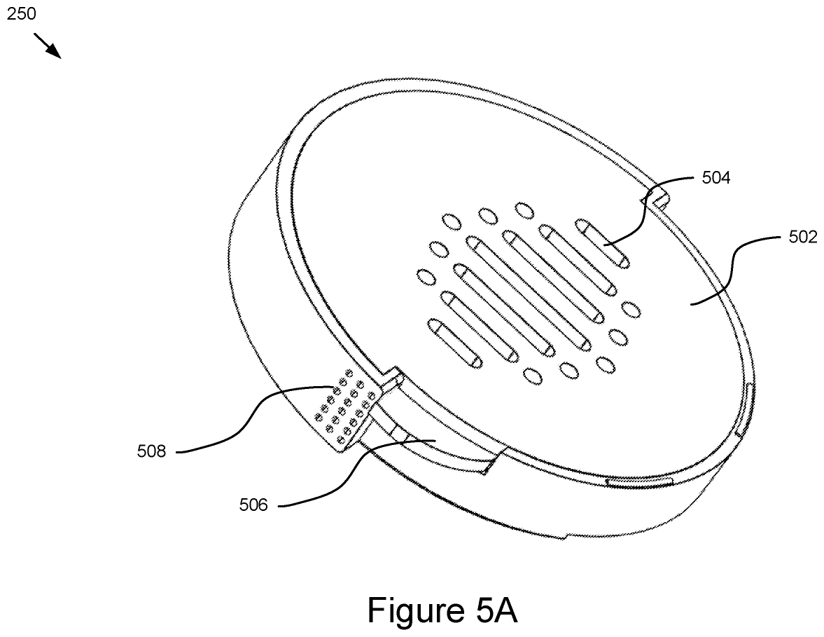

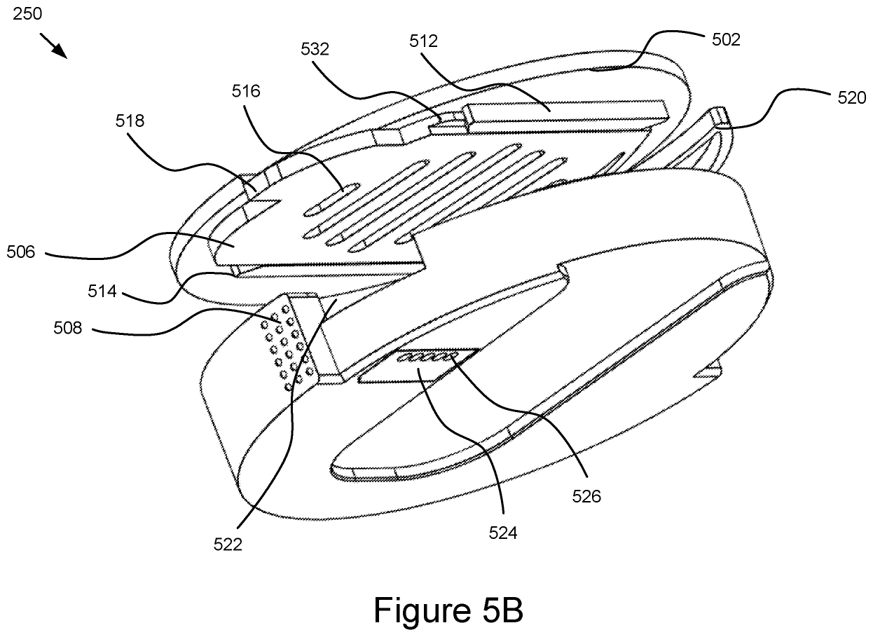

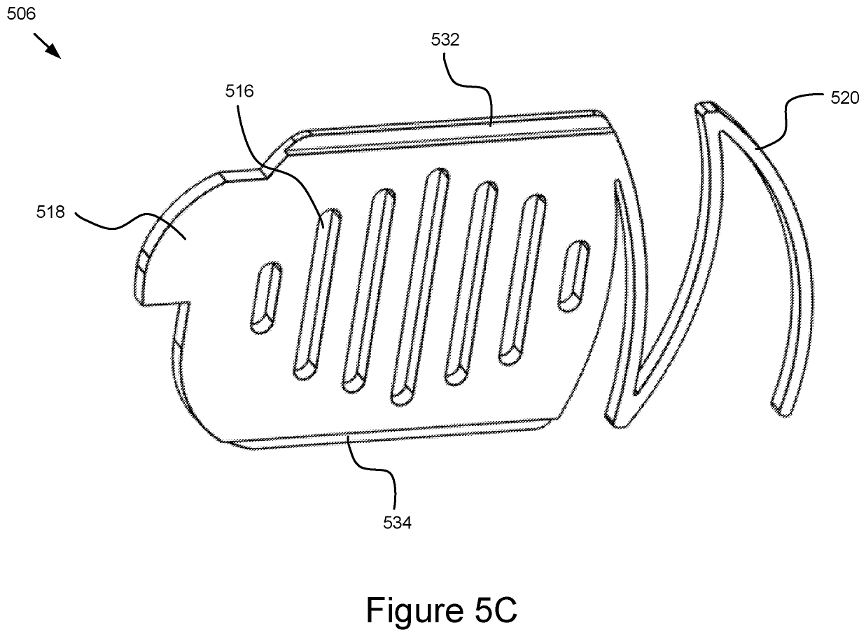

FIGS. 5A-5B depict various views of an example scent cartridge 250 for the example vehicle scent dispensing device 132. The example scent cartridge 250 depicted in FIG. 5A includes a lid 502 having scent openings 504 and a sliding plate valve 506. In some implementations, scent cartridge 250 includes protrusions 508 on the sides to provide additional grip for handling the scent cartridge 250.

FIG. 5B depicts an exploded view of the example scent cartridge 250 depicted in the example of FIG. 5A, according to some implementations. FIG. 5B shows the sliding plate valve 506 slidably coupled with the lid 502 by retainers 512 and 514. Sliding plate valve 506 includes spring 520, activation tab 518, and scent openings 516. The scent cartridge 250 in the example of FIG. 5B also includes a scent cartridge identification component 524 (e.g., an identification chip) having electrical contacts 526. As described elsewhere herein, the vehicle scent dispensing device 132 may include spring-loaded pins that create an electrical connection with the electrical contacts 526 of the scent cartridge 250 such that the controller 188 can determine the characteristics of scent cartridge 250 (e.g., the scent contained in scent cartridge 250, the amount of time scent cartridge 250 has been installed in the vehicle scent dispensing device 132, the amount of time the scent cartridge 250 has been active, etc.) and use the characteristics of scent cartridge 250 to determine a scent diffusion level.

FIG. 5C depicts another view of an example sliding plate valve 506. FIG. 5C shows tabs 532 and 534 configured to engage with retainers 512 and 514 to hold the sliding plate valve in contact with the lid 502. When lid 502 is in place on the scent cartridge 250, spring 520 engages with an interior wall of the scent cartridge 250 and pushes the sliding plate valve toward opening 522 where the activation tab 518 protrudes from the scent cartridge 250. When outside of the vehicle scent dispensing device 132, the spring holds the sliding plate valve in a closed position such that the sliding plate covers the scent openings 504 in the lid 502 and no scent can escape scent cartridge 250. When scent cartridge 250 is inserted into the vehicle scent dispensing device 132 the activation tab 518 is depressed and spring 520 compresses such that the body of the sliding plate valve 506 slides toward the spring side and scent openings 516 align with the scent openings 504 in the lid 502 allowing scent to diffuse from the scent cartridge 250.

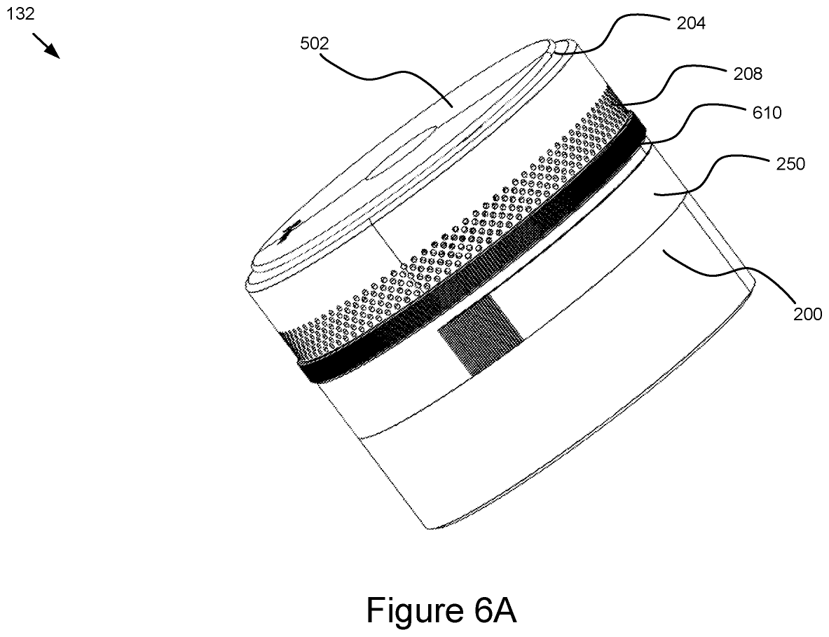

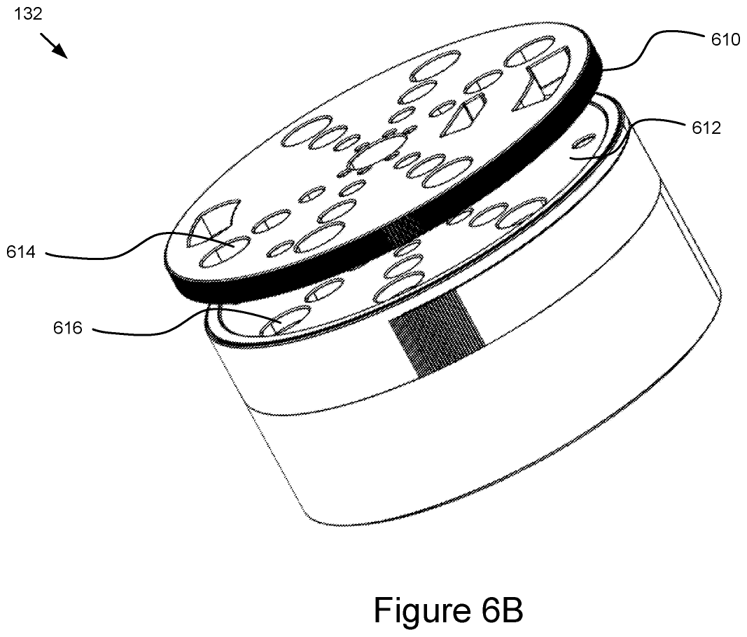

FIGS. 6A-6B depict the vehicle scent dispensing device 132 according to another embodiment. As shown in FIG. 6A, the housing 200 includes a rotatable plate valve 610 between the scent cartridge compartment and the fan compartment. In some implementations, the rotatable plate valve 610 may rotate about a bearing and may include various hole patterns. The hole patterns allow air to flow through the holes and can have various configurations to allow different amounts of air through the surface areas of the holes. The rotatable plate valve 610 may be rotatable within the housing 200 and may rotate between an open position, a closed position, and/or various partially open positions to regulate an airflow pathway to the scenting agent of scent cartridge 250. When the rotatable plate valve 610 is rotated into the open position the rotatable plate valve 610 allows for various holes within the plate body to line up with holes in a top section of a scent cartridge chamber where the scent cartridge 250 is installed. Air is allowed to then pass over the scent cartridge 250 and through the various holes within the rotatable plate valve 610 to allow for scent to be diffused. When the rotatable plate valve 610 is rotated into the closed position, the various holes of the rotatable plate valve 610 do not line up with the holes of the scent cartridge chamber which effectively seals off the scent cartridge 250 to preserve the life of the scent cartridge 250 when it is not needed. The vehicle scent dispensing device 132 may include a fan compartment housing a fan and/or fan motor that can be turned on and off to pull air through perforations 208 and over the scent cartridge 250 and out of the recess 204 when the rotatable plate valve 610 is in the open position.

FIG. 6B depicts a partially exploded view of vehicle scent dispensing device 132. In the example of FIG. 6B, the fan compartment is not shown to allow for viewing of the rotatable plate valve 610 and the top of the scent cartridge chamber 612. As described above, the rotatable plate valve 610 and the top of scent cartridge chamber 612 have holes 614 and 616 that allow for scent to flow from the scent cartridge chamber when the holes are at least partially aligned.

In some embodiments, as depicted in the example of FIGS. 6A and 6B, the rotatable plate may be manually turned by a user to open or close the scent cartridge chamber. In other embodiments, the vehicle scent dispensing device 132 may include a servo assembly (not shown) that is included within the housing 200. In such an embodiment, the rotatable plate valve 610 may be opened and closed by the servo assembly. In an open configuration, the servo assembly rotates the rotatable plate valve 610 using rods that extend out of the top of the scent cartridge chamber 612 until the holes 614 in the rotatable plate valve 610 line up with the holes 616 in the top of the scent cartridge chamber 612. The servo assembly may be connected to the power supply 185 and/or the controller 188 and when a signal is sent to open or close the device scent cartridge chamber, the servo assembly may cause the rotatable plate valve 610 to transition between the open to the closed positions. In some implementations, the servo assembly can also include various partially open settings to adjust for the amount of air flow that passes through the gap between the holes in the rotatable plate valve 610.

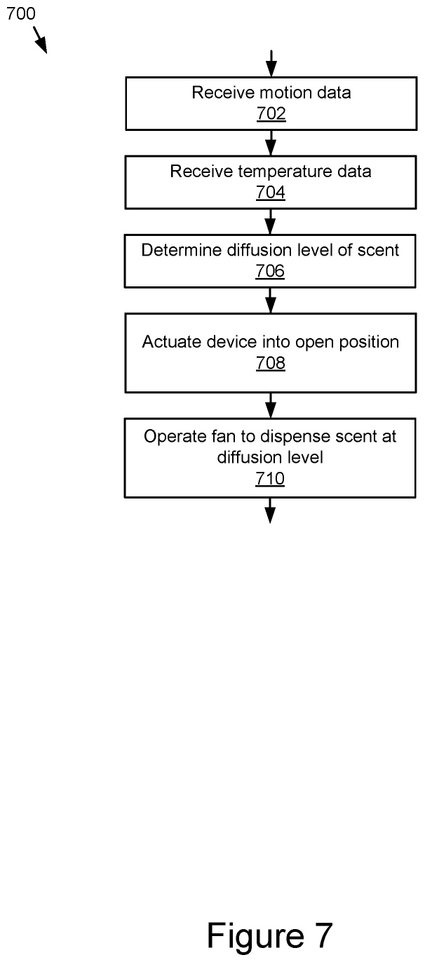

FIG. 7 is a flowchart of an example method 700 for dispensing a scent using a vehicle scent dispenser 132. At 702, the controller 188 receives motion data from the sensors 186, such as an accelerometer or other motion sensor. The motion data relates to a movement of the vehicle 158 that contains the vehicle scent dispensing device 132. The controller 188 uses the motion data to determine whether the vehicle 158 is being operated and moving and, if so, to cause the scent dispensing device 132 to begin operation. By analyzing the motion data, the controller 188 can cause scent to only be diffused when the vehicle 158 is moving, which indicates that a user 112 is inside of the vehicle. Alternatively, when the motion data indicates that the vehicle is not moving, the controller 188 can determine if the lack of movement is over a short period of time, e.g., the car is at a stop light and was actively moving prior to that stop, or if the vehicle has not moved for a longer period of time, such as a few hours, which indicates that the vehicle 158 is parked and scent should not be diffused. When the motion data indicates that the vehicle 158 is parked and/or unattended, the controller 188 causes the scent cartridge 250 to be sealed off from airflow, such as by using the valve 312, sliding plate valve 506, or plate valve 610. Sealing off the scent cartridge 250 keeps the scenting agent within the cartridge and preserves the scent for longer as compared to scent dispensers that do not seal off the scent cartridge.

At 704, the controller 188 receives temperature data from the sensors 186, such as a temperature sensor. The temperature data reflects the current and/or historical temperatures of the interior of the vehicle 158. The temperature data is useful, as the scenting agent of the scent cartridge 250 provides more scent in warmer temperatures than in colder temperatures. The temperatures of the interior of the car can swing from various hot and cold temperatures depending on the weather, outside temperature, and/or the interior AC or heat temperature that is set by the user 112. The controller 188 uses the current temperature to determine settings for the valve and/or the fan speed. At 706, the controller 188 determines a diffusion level using both the motion data and the temperature data. The diffusion level is a setting of the device to control how much scent and/or when scent is emitted into the vehicle 158. The motion data is used by the controller 188 to determine when the vehicle 158 is being operated and scent should be emitted, and the temperature data is used by the controller 188 to determine how much airflow should be passed through the device 132 and how high and/or what duration the fan speed should operate at. The controller 188 may further factor in various user preference settings that can be retrieved from the dispenser management application 160, such as whether a user prefers a strong scent or a milder scent to preserve the scent cartridge 250 for longer. By determining a diffusion level of the scent, the controller 188 can preserve the life of the scent cartridge by only allowing airflow when the vehicle 158 is being operated and can match the airflow output to the environmental factors, e.g., causing the fan to blow more in the colder months and less in the summer months to achieve optimal scent levels. Essentially, the controller 188 can create a stable and consistent scenting experience for the user 112 using the motion and temperature data to determine a scent diffusion level. Additionally, in some implementations, the controller can determine when high temperatures are detected and cause the fan and/or other components to auto shutoff if it would risk damaging the power supply or the batteries of the device 132. By detecting and shutting off the device 132 in high heat, it can preserve the life of the batteries, the lifetime of the scent cartridge 250, and/or reduce a risk of fire or damage to the device 132.

In some implementations, the diffusion level may be also based on a user setting, such as a low level, medium level, or high level that may be input by the user 112 to set a scent preference. In some implementations, the user setting can be changed by providing input on the user device 106, while in further implementations an input device may be used, such as a button or slider, to adjust the user setting. In some implementations, the user settings may also include various modes, such as burst modes, wind down modes, etc. and the diffusion level can be further changed based on the selected modes. In some implementations, the output device 192, such as a row of lights, etc., may signal to the user 112 the selected settings and diffusion level.

At 708, the controller 188 controls the valve to allow airflow to pass over the scenting agent of the scent cartridge 250 and out of the device 132 as described above. In some implementations, the controller 188 can actuate the rotatable plate valve 610 to a determined position as needed based on the determined diffusion level of the scent. In some implementations, the controller can cause the fan 190 to operate at a specific speed to create an amount of pressure that opens the valve 312 based on the determined diffusion level of the scent.

At 710, the controller 188 causes the fan 190 to blow air over the scenting agent and out of the device 132 in order to diffuse the scent within the vehicle 158. The fan 190 allows the scent to be diffused more quickly than just having airflow naturally pass over the scenting agent of the scent cartridge 250. Using the fan 190, the scent can be quickly dispersed into the vehicle and is more noticeable to the user 112. This allows the device 132 to quickly emit the scent when it detects that the vehicle 158 is in motion (e.g., a user 112 is within the vehicle 158) and the device 132 has less of a warm-up period where the scent is being diffused but is not yet noticeable to the user 112 as compared to conventional car scent devices. In some implementations, the fan 190 operates continuously as the vehicle 158 is being used. In further implementations, the device 132 can operate the fan on various pulses in order to reduce the effect of nose blindness, allowing the scent to diffuse and then settle while the fan is off for a period of time before another pulse of the fan emits another burst of scent for a period of time.

In some implementations, the vehicle scent dispensing device 132 can auto shutoff and seal the airflow to the scent cartridge 250. By auto shutting off the device and sealing the scent cartridge 250, the lifetime of the scent is increased when sealed off while not in use and the user 112 does not have to manage the opening or closing of the device 132 manually. For example, in some implementations, when the vehicle scent dispensing device 132 connects to the user device 106, such as by a Bluetooth connection or detects a connection within a geofence, the device 132 may open and being preparing for scent dispensation. In some implementations, the device 132 can specifically look for the Bluetooth beacon from the user device 106 to indicate when to operate or shutoff. In some implementations, the vehicle 158 itself may act as a signal blocker, similar to a Faraday cage, and the beacon signals are only detectable at strong enough levels when the user device 106 is within the vehicle 158, indicating that the user 112 is in the vehicle 158 and scent should be emitted.

In further implementations, after a connection between the device 132 and the user device 106 is lost, such as by a user moving out of Bluetooth range of the device 132, the device 132 will auto shutoff and seal the airflow to the scent cartridge 250. In other implementations, the auto shutoff operates based on detected motion from a sensor 186. For example, when the motion is detected (e.g., the vehicle 158 is moving) the device 132 opens up the airflow and begins dispensing scent. However, when motion is not detected for a period of time, the device 132 auto shutoff is enacted and the airflow to the scent cartridge 250 is sealed. In further implementations, the temperature data can be used to enact the auto shutoff. For example, if the temperature data indicates that a user 112 has begun using the vehicle 158, such as a change in interior temperature from too cold to warm, indicating the heater is running or too hot to cold indicating the AC is running, then the device 132 may begin diffusing scent. When the temperature data indicates a change from a comfortable temperature to either too hot or too cold, then an auto shutoff is enacted.

In some implementations, the device 132 may be used separate from a vehicle 158, where the device 132 instead operates as a personal scent dispenser and is capable of emitting a scent within a space (such as an eight-foot area) around a user 112. The user 112 can portable move the device 132 around and rely on the battery power to cause the fan to emit the scent as needed. This allows a user 112 to take the device 132 with them and enjoy a stable scent experience as they move around that may reduce and/or increase the fan speed or opening size based on the amount of motion detected and/or the temperature data.

The foregoing description, for purpose of explanation, has been described with reference to various embodiments and examples. However, the illustrative discussions above are not intended to be exhaustive or to limit the invention to the precise forms disclosed. Many modifications and variations are possible in view of the above teachings. The various embodiments and examples were chosen and described in order to best explain the principles of the invention and its practical applications, to thereby enable others skilled in the art to utilize the innovative technology with various modifications as may be suited to the particular use contemplated. For instance, it should be understood that the technology described herein can be practiced without these specific details in some cases. Further, various systems, devices, and structures are shown in block diagram form in order to avoid obscuring the description. For instance, various implementations are described as having particular hardware, software, and user interfaces. However, the present disclosure applies to any type of computing device that can receive data and commands, and to any peripheral devices providing services.

In some instances, various implementations may be presented herein in terms of algorithms and symbolic representations of operations on data bits within a computer memory. An algorithm is here, and generally, conceived to be a self-consistent set of operations leading to a desired result. The operations are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the following discussion, it is appreciated that throughout this disclosure, discussions utilizing terms such as “processing,” “computing,” “calculating,” “determining,” “displaying,” or the like, refer to the action and methods of a computer system that manipulates and transforms data represented as physical (electronic) quantities within the computer system's registers and memories into other data similarly represented as physical quantities within the computer system memories or registers or other such information storage, transmission or display devices.

A data processing system suitable for storing and/or executing program code, such as the computing system and/or devices discussed herein, may include at least one processor coupled directly or indirectly to memory elements through a system bus. The memory elements can include local memory employed during actual execution of the program code, bulk storage, and cache memories that provide temporary storage of at least some program code in order to reduce the number of times code must be retrieved from bulk storage during execution. Input or I/O devices can be coupled to the system either directly or through intervening I/O controllers. The data processing system may include an apparatus may be specially constructed for the required purposes, or it may comprise a general-purpose computer selectively activated or reconfigured by a computer program stored in the computer.

The foregoing description has been presented for the purposes of illustration and description. It is not intended to be exhaustive or to limit the specification to the precise form disclosed. Many modifications and variations are possible in light of the above teaching. It is intended that the scope of the disclosure be limited not by this detailed description, but rather by the claims of this application. As will be understood by those familiar with the art, the specification may be embodied in other specific forms without departing from the spirit or essential characteristics thereof. Likewise, the particular naming and division of the modules, routines, features, attributes, methodologies and other aspects may not be mandatory or significant, and the mechanisms that implement the specification or its features may have different names, divisions, and/or formats.

Furthermore, the modules, routines, features, attributes, methodologies and other aspects of the disclosure can be implemented as software, hardware, firmware, or any combination of the foregoing. The technology can also take the form of a computer program product accessible from a computer-usable or computer-readable medium providing program code for use by or in connection with a computer or any instruction execution system. Wherever a component, an example of which is a module or engine, of the specification is implemented as software, the component can be implemented as a standalone program, as part of a larger program, as a plurality of separate programs, as a statically or dynamically linked library, as a kernel loadable module, as firmware, as resident software, as microcode, as a device driver, and/or in every and any other way known now or in the future. Additionally, the disclosure is in no way limited to implementation in any specific programming language, or for any specific operating system or environment. Accordingly, the disclosure is intended to be illustrative, but not limiting, of the scope of the subject matter set forth in the following claims.

What is claimed is:

1. A scent dispensing device, comprising:
a scent cartridge configured to contain a scenting agent;
a controller configured to determine a scent diffusion level of the scenting agent;
a valve, wherein the controller is configured to operate the valve to regulate an airflow pathway to the scenting agent based on the scent diffusion level; and
a fan, wherein the controller is configured to control the fan to draw air through the airflow pathway and over the scenting agent based on the scent diffusion level.

2. The scent dispensing device of claim 1, wherein the valve is a rotatable plate valve.

3. The scent dispensing device of claim 2, wherein the rotatable plate valve comprises:
a plate body having a plurality of plate body holes configured to at least partially align with a plurality of scent cartridge chamber holes in the scent dispensing device to expose the scenting agent.

4. The scent dispensing device of claim 3, further comprising:
a servo assembly, wherein the servo assembly is controlled by the controller to operate the rotatable plate valve.

5. The scent dispensing device of claim 1, wherein the scent cartridge comprises the valve.

6. The scent dispensing device of claim 5, wherein the valve is an umbrella valve coupled with the scent cartridge.

7. The scent dispensing device of claim 6, wherein the controller is configured to operate the umbrella valve by controlling the fan to create a pressure differential between the scent cartridge and a fan compartment housing the fan.

8. The scent dispensing device of claim 5, wherein the valve is a sliding plate valve.

9. The scent dispensing device of claim 8, wherein the sliding plate valve comprises:
a sliding plate having a plurality of plate scent openings;
a spring coupled with a first edge of the sliding plate; and
an activation tab coupled with a second edge of the sliding plate, the second edge opposite the first edge, wherein the activation tab is configured to engage with a housing of the scent dispensing device when the scent cartridge is inserted into the scent dispensing device and compress the spring such that the plurality of plate scent openings align with a plurality of scent cartridge scent openings in a lid of the scent cartridge.

10. The scent dispensing device of claim 9, wherein the spring is configured to return the sliding plate to a closed position when the scent cartridge is removed from the scent dispensing device such that the sliding plate covers the plurality of scent cartridge scent openings in the lid of the scent cartridge.

11. A scent dispensing device, comprising:
a housing;
a scent cartridge configured to contain a scenting agent, the scent cartridge comprising:
a lid having a first plurality of openings;
a plate slidably coupled with the lid, the plate having a second plurality of openings;
a spring coupled with a first edge of the plate; and
an activation tab coupled with a second edge of the plate, the second edge opposite the first edge, wherein the activation tab is configured to engage with the housing when the scent cartridge is inserted into the scent dispensing device and compress the spring such that the second plurality of openings aligns with the first plurality of openings;
a controller configured to determine a scent diffusion level of the scenting agent; and
a fan, wherein the controller is configured to control the fan to draw air through an airflow pathway and over the scenting agent based on the scent diffusion level.

12. The scent dispensing device of claim 11, wherein the housing includes a plurality of perforations configured to allow air from outside of the scent dispensing device to enter the airflow pathway.

13. The scent dispensing device of claim 11, wherein the housing includes a recess configured to allow air scented air from the airflow pathway to exit the scent dispensing device.

14. The scent dispensing device of claim 11, further comprising:
a motion sensor configured to capture motion data; and
a temperature sensor configured to capture temperature data;
wherein, the controller is configured to determine the scent diffusion level based on the motion data and the temperature data.

15. The scent dispensing device of claim 14, wherein the controller is further configured to determine the scent diffusion level based on user input.

16. The scent dispensing device of claim 14, wherein the scent cartridge further comprises a scent cartridge identification component, wherein the controller can identify characteristics of the scent cartridge by reading the scent cartridge identification component and determine the scent diffusion level based on the characteristics of the scent cartridge.

17. A scent cartridge for a scent dispensing device, comprising:
a lid having a first plurality of openings;
a plate slidably coupled with the lid, the plate having a second plurality of openings;
a spring extending from a first edge of the plate; and
an activation tab extending from a second edge of the plate, the second edge opposite the first edge, wherein the activation tab is configured to engage with a housing of the scent dispensing device and compress the spring such that the second plurality of openings aligns with the first plurality of openings.

18. The scent cartridge for a scent dispensing device of claim 17, further comprising a scenting agent.

19. The scent cartridge for a scent dispensing device of claim 18, wherein the scenting agent comprises a gel.

20. The scent cartridge for a scent dispensing device of claim 19, further comprising a scent cartridge identification component, wherein a controller of the scent dispensing device can identify characteristics of the scent cartridge by reading the scent cartridge identification component.
